(19) United States
(12) Patent Application Publication
(10) Pub. No.: US 2009/0123399 A1
(43) Pub. Date: May 14, 2009

(54) Packaged liquid edible composition comprising carotenoids

(75) Inventors: Marnix P van Amerongen, AT Vlaardingen (NL); Gail Jenkins, Sharnbrook (GB); Julia Sarah Rogers, Sharnbrook (GB); Gila Seewi, Heilbronn (DE)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc. d/b/a Unilever

(21) Appl. No.: 12/291,591

(22) Filed: Nov. 12, 2008

(30) Foreign Application Priority Data
Nov. 12, 2007 (EP) EP07120470

(51) Int. Cl. A61K 8/31; A61K 31/015; A61K 31/047; A61K 31/01

(52) U.S. Cl. 424/59; 514/763; 514/729; 514/762; 424/63

(57) ABSTRACT

A packaged liquid edible composition comprising a blend of at least two species of fruit and/or vegetables in liquified, puree, pulp, concentrate, extract or juice form, wherein said composition is packaged in a container in an amount of a specific volume and having a specific consistency, and wherein said composition comprises carotenoids.

### FIELD OF THE INVENTION

The invention relates to a packaged liquid edible composition for treating the human skin by ingestion comprising a blend of at least two species of fruit and/or vegetables in liquified, puree, pulp, concentrate, extract or juice form, wherein said composition is packaged in a container in an amount of a specific volume and having a specific consistency, and wherein said composition comprises carotenoids.

### BACKGROUND OF THE INVENTION

Until recently, with some possible exceptions, all skin care products (i.e. products to increase the health, condition and/or appearance of the skin, the latter e.g. with respect to preventing the human skin from signs of aging) were compositions for topical application containing one or more actives: skin creams, lotions and the like that had to be applied to the skin. Such topical products have disadvantages, e.g. like a leaving a fatty feel, reaching only the external layers of the skin, and having an effect only on the area's on which the skin cream was applied (e.g. an uneven treated look).

Since recently, there is growing insight that one can also treat, condition, or improve the human skin from inside: by consuming an edible composition containing one or more active components that are taken up and which are transported to the skin. Such clearly has advantages in terms of the skin on the whole body being treated, and leaving no fatty feel to the skin. The active components are not necessarily the same as when applied topical: something needs to be taken up and transported to the skin, and not harmful when ingested. An example of ingestible compounds that can be actives for the skin are carotenoids.

DE 19852536 discloses drinks comprising fruit and vegetable components, further enriched by vitamin E (next to containing vitamins A and C), which drinks have a Brix value of more than 11.2°, and which drinks comprise per 100 g more than 120 μg, preferably more than 400 μg vitamine A or more than 720 μg, preferably more than 2400 μg pro-vitamine A, as well as at least 9 mg, preferably at least 30 mg vitamin C, and 1.5 mg, preferably at least 5 mg vitamin E, and which drinks are stated to have an increased mixing- and turbidity stability, as well as being able to provide natural vitamins. These drinks are reported to contain per 100 ml at least 50% of the RDA of vitamine A (as pro-vitamine A), vitamine C and vitamine E.

### SUMMARY OF THE INVENTION

There is a need for a composition which, when ingested (by human adults) in the proper amounts, for a prolonged period (e.g. 6 weeks, preferably at least 12 weeks, taken daily) can serve to deliver carotenoids to the skin, preferably in such a way as to give a mild colouring effect (for aesthetic reasons and/or to provide an indication of delivery of the right dose of carotenoids), e.g. a slight orange and/or yellow and/or brownish colouring of the skin. It is also desired that such composition is provided in such a way that chances on high intakes of said composition are low, as high intake may lead to undesired yellowing or orange colouring of the skin (i.e. too intense colouring), which may have an adverse effect on the perceived quality of the product as a “beauty product”. For topical skin care products, there is a “natural” limit to what can or is being applied to the skin, in that too much of a topical skin care product will leave a fatty feel or look, and additionally one can usually directly see the result of the application, causing people to stop applying when the desired effect is achieved, to avoid an “over effect”. Such natural limit is less the case for skin care products which need to be ingested: no fatty look or feel is left after ingestion, and secondly there is a lag time between taking in the product and the effect on the skin. In other words: people may be tempted to consume (eat, drink) more of the ingestible composition in their desire to enhance their skin condition and/or appearance. When such is done with a composition containing carotenoids, after some time too much colouring can be the result, which in the end may provide the consumers with a negative perception of the product.

Hence, there is a desire for an ingestible skin care product, preferably something perceived as a food product (or possibly a food additive, but preferably a food product) rather than a medicament or chemical, which product may be able to deliver carotenoids to the skin, and preferably which gives (in particular to people of Caucasian origin, preferably with fair skin) a mild colouring (mild yellow and/or orange and/or brownish colouring or hue of the skin) upon prolonged (e.g. at least 12 weeks) and regular (e.g. daily or almost daily, like at least 5 times a week) consumption, and which product is preferably designed such that consumers are likely to be inclined not to consume such an amount of the ingestible skin care product which could lead to an over-intense yellow and/or orange and/or brownish colouring (over-intense as in the opinion of the consumer concerned). Preferably, the mild colouring as set out above is such that in a test of oral administration over a prolonged period (e.g. minimal of 12 weeks) of the ingestible skin care product, carotenoid levels within the skin can be increased compared to a placebo, as measured by a significant increase (with a p-value of <0.05) in chromameter b* value. Preferably, the colouring is (when measured at the palm at the hand) when measured using a Chromameter, the increase (with a p-value of <0.05) in chromameter b* value is at least 0.5, preferably at least 1. Preferably, the colouring is not too strong, in that, when measured using a Chromameter, the increase (with a p-value of <0.05) in chromameter b* value is less than 2.5, preferably less than 2 (as compared to a placebo, when measured under the conditions as referred to above), and wherein chromameter a* and L value are not (statistically) significantly different when compared to a placebo. For this, the chromameter readings taken are preferably of the palm of the hand, as usually such is the area of the skin that shows the largest increase in chromameter b* value.

It has now been found that the above may be achieved (at least in part) by a packaged liquid edible composition for treating the human skin by ingestion, said composition comprising a blend of at least two species of fruit and/or vegetables in liquified, puree, pulp, concentrate, extract or juice form, wherein said composition is packaged in a container in an amount of 50 to 150 g, and wherein said packaged composition comprises per container carotenoids in an amount of 10 to 14 mg, and which composition has a viscosity of 10 to 100 mPa s. In this, the viscosity an be measured using a rheometer like the Bohlin Rheometer CVO, using geometry C25, and the above data are for measuring the sample at 10° C. and at a shear rate of 50 s⁻¹.

### DETAILED DESCRIPTION OF THE INVENTION

Except in the examples, or where otherwise explicitly indicated, all numbers in this description indicating amounts of material or conditions of reaction, physical properties of materials and/or use are to be understood as modified by the word “about.” All amounts are by weight, unless otherwise specified.

It should be noted that in specifying any range of concentration or amount, any particular upper concentration can be associated with any particular lower concentration or amount.

For the avoidance of doubt, the word “comprising” is intended to mean “including” but not necessarily “consisting of” or “composed of.” In other words, the listed steps or options need not be exhaustive.

When it is referred to herein as “carotenoid” or “carotenoids”, it is to be understood as to comprise one or more of lycopene, beta-carotene, lutein, astaxanthin, zeaxanthin, alpha-carotene, beta-cryptocanthin, canthaxanthin.

It is believed that by providing the product in the form of a fruit and/or vegetable-comprising liquid having both a high viscosity (yet still being drinkable) and being in the form of a limited amount, such liquid is still a food product, but not seen as a thirst quencher, and following that less likely of having a tendency of being consumed in such amounts that a colouring is achieved which may be perceived as too intense by the consumer or people in his or her surrounding. The fruit and or vegetables can provide both the desired thickness or consistency and part or all of the carotenoids. It is also believed that by using a blend of at least two species of fruits and/or vegetables (one fruit, one vegetable, two species of fruit, two species of vegetables) also a flavour can be obtained which does not directly provide a link to known fruit or vegetable juices, which also makes it less likely to have a tendency of being seen as a thirst quencher, and so helping to avoid “overdosing” in the way set out above.

In the composition according to the present invention, the total level of carotenoids is preferably 10 to 13 mg, more preferably 11 to 12 mg. In the composition according to the invention, it is preferred that it comprises β-carotene in an amount of 5 to 8 mg, preferably 6 to 7.5 mg. This is to be understood as part of the total amount of 10 to 13 mg. In addition to or next to said level of β-carotene, it is preferred that the composition according to the present invention comprises lycopene in an amount of 2 to 12 mg, preferably in an amount of 2.5 to 8 mg (again as part of the total amount of carotenoids of 10 to 13 mg). In addition to or next to said level of β-carotene and/or lycopene, it is preferred that the composition according to the present invention comprises lutein in an amount of 1 to 3 mg.

In a preferred way, the composition according to the present invention preferably comprises:
β-carotene in an amount of 5.5 to 8 mg, preferably 6.5 to 7.5 mg, and
lycopene in an amount of 2 to 4 mg, preferably 2.5 to 3.5 mg, and
lutein in an amount of 1 to 3 mg, preferably 1.5 to 2.5 mg.

In the present invention, it is preferred that the composition comprises puree and/or juice and/or concentrate and/or juice concentrate of fruit and/or vegetable origin in an amount corresponding to 200-400 g, preferably 250-350 g fresh weight of fruit and/or vegetables. Reason for such is that it can both provide all or part of the carotenoids, and when the fruits and/or vegetables are (at least part) in the form as set out in this paragraph, it can also conveniently provide the desired thickness and/or consistency and/or viscosity. The composition according to the present invention preferably also comprises insoluble solids of fruit and/or vegetable origin, and preferably in an amount corresponding to 200-400 g, preferably 250-350 g fresh weight of fruit and/or vegetables.

In the composition according to the present invention, it is preferred that the composition has a viscosity of 15 to 90, more preferably 20 to 60 mPa s, to further distinguish it from ordinary juices so that it is not likely to be seen as a thirst quencher. When the consistency is measured using the method according to DIN standard DIN EN ISO 2431 (flow method), the consistency is preferably such that using a 4 mm flow cup, the time measured is between 12 to 25 seconds, preferably 13 to 20 seconds.

To further help setting the present composition apart from normal fruit or vegetable juices it is preferred that said composition has a Brix value of 14° to 21°, preferably 15.8° to 19.4°. By doing so, the composition, especially if it comprises puree and/or juice and/or concentrate and/or juice concentrate of fruit and/or vegetable origin in an amount corresponding to 200-400 g, preferably 250-350 g fresh weight of fruit and/or vegetables, is perceived as “rich”, in fact too rich to be perceived as a thirst quencher. Ordinary fruit juices like apple juice usually have a Brix value of about 10-11°.

It may be preferred that the composition according to the present invention is packaged in a container in an amount of 75 to 125 g, more preferably 90-110 g. The small volume concerned also may help setting it apart from e.g. normal fruit and/or vegetable juices, so that over-dosing is less likely to occur.

The composition according to the present invention may further comprise additional actives which are beneficial (when consumed) to the skin. Hence, it may be preferred that the composition according to the present invention further comprises vitamine C, in an amount of 50 to 150 mg, preferably 55 to 120 mg, more preferably 60 to 100 mg. Likewise, it may be preferred that said compositions (with or without vitamin C) further comprises vitamine E in an amount of 8 to 12 mg, preferably 9 to 11 mg. Likewise, it may be preferred that said compositions (with or without vitamin C and/or E) further comprises zinc in an amount of 3 to 7 mg, preferably in an amount of 4 to 6 mg. When it is referred to herein as “vitamin C”, it is to be understood as to comprise also one or more of its salts and precursors, such as ascorbic acid and salts of ascorbic acid and/or sources thereof. When it is referred to herein as “vitamin E”, it is to be understood as to comprise also one or more of various forms of vitamin E, like tocopheryl acetate, and more specifically d-α-tocopheryl acetate, as well as sources of these compounds.

In the composition according to the present invention, e.g. as a source of all or part of the carotenoids like β-carotene, it may be preferred that it comprises orange carrot in liquified, puree, pulp, concentrate, extract or juice form, in an amount corresponding to fresh orange carrot of 40 to 200 g fresh weight, preferably 50 to 150 g fresh weight. Similarly, it may be preferred that it comprises purple carrot in liquified, puree, pulp, concentrate, extract or juice form in an amount corresponding to fresh purple carrot of 20 to 150 g fresh weight, preferably 30 to 100 g fresh weight.

In order to get a good taste and/or aroma and/or as source of additional actives like vitamin C, the composition according to the present invention may further comprise a blend of at least one species of fruit in liquified, puree, pulp, concentrate, extract or juice form and at least one species of vegetables in liquified, puree, pulp, concentrate, extract or juice form. In a suitable way, the compositions may comprise one or more of (part or all of) apple, white grape, red grape, banana, acerola cherry, lemon, orange, grapefruit, in liquified, puree, pulp, concentrate, extract or juice form.

The invention further relates to the a method for treating the human skin by ingestion of a composition according to the above. In this case, the “human” is preferably a human of Caucasian race, and/or preferably has a skin of type category I, II, or III, of the Fitzpatrick skin types categorisation. The above referred “treating of the skin” preferably comprises one or more of skin care, skin condition, skin appearance, skin colour, for a skin bronzing, skin browning, skin colouring effect, UV-protection, UV-B-protection, sun-screen, protection against aging, protection against photo-induced aging, providing anti-oxidants to the skin (e.g. but not exclusively to the dermis). Preferably, the use referred herein is when the composition is ingested regularly (preferably once every two days, preferably at least 5 days a week, more preferably daily) and for a prolonged time (for at least 4 weeks, preferably at least 8 weeks, more preferably at least 12 weeks).

### EXAMPLES

### Examples 1 and 2

The compositions of examples 1 and 2 in table 1 were prepared, and bottled in 100 ml bottles, the total composition having a weight per bottle of approx. 107 g (corresponding to a volume of about 100 ml).

TABLE 1
composition examples 1, 2 (in absolute weight) and 3 (in weight %)

| | Example 1, | | Example 2, | | Example 3, |
|---|---|---|---|---|---|
| Ingredient | amount in g | | amount in g | | amount wt % |
| Acerola puree | 1.03 | | 0.93 | | 0.94 |
| Banana puree | 8.22 | | 7.42 | | 8.50 |
| Red grape juice concentrate | 8.73 | | 7.88 | | 6.61 |
| Lemon juice | 0.10 | | 0.09 | | 0.09 |
| Rose hips puree | 6.16 | | 4.64 | | 4.72 |
| Elderberries | 1.54 | | 1.39 | | 1.13 |
| Flower-infused apple juice | 4.62 | | 3.25 | | 6.61 |
| extract | | | | | |
| Carrot juice concentrate | 11.09 | | 10.02 | | 10.30 |
| “Carotan” | | | | | |
| Carrot juice concentrate | 0 | | 6.03 | | 0 |
| “Lycopene” | | | | | |
| Carrot juice concentrate | 1.03 | | 0.93 | | 1.42 |
| “Purple” | | | | | |
| Lycopene-rich tomato extract | 2.67 | | 0 | | 2.46 |
| (Lyc- O-Mato Red, | | | | | |
| ex Lycored) | | | | | |
| Vitamine mix with lutein | 0.30 | | 0.30 | | 0.28 |
| Demin water | 61.63 | | 64.93 | | 56.72 |
| Flavours | 0 | | 0 | | 0.22 |
| Total | 107.12 | g | 107.81 | g | 100% |

The vitamine mix with lutein was composed of (per g): 36.667 mg tocopherol equivalent (vitamin E), 300 mg vitamine C, 7 mg lutein, 16.67 mg zinc, 0.055 mg selenium. The carrot juice concentrate “lycopene” was Nutrired, from Ernteband. The lycopene-rich tomato fraction contained about 1500 ppm (±100) ppm lycopene. The compositions were dark-red-brownish, fairly viscous, non-clear products, of about 17-17.6° Brix. Example 1 had a viscosity about 27 mPa s, by measuring with a Bohlin Rheometer CVO, using geometry C25, and measuring the sample at 10° C. and at a shear rate of 50 s⁻¹. Example 2 was not measured but appeared to have a similar consistency.

The compositions of examples 1 and 2 both contained approximately 7 mg β-carotene, 3 mg lycopene, 2 mg lutein, 60 mg vitamin C, 10 mg vitamin E.

### Example 3

The composition of example 3 is also given in table 1 above. This composition is used in a study (randomised, double blind, against a placebo). The composition of the placebo is set out below in table 2. The appearance of the placebo matched in appearance (consistency, texture, colour, flavour, aroma) that of the example to be used in the study. The composition of example 3 had a similar appearance (e.g. colour) as for examples 1 and 2, and contained approximately the same levels of the various carotenoids mentioned and vitamins E and C. Example 3 had (like example 1) a viscosity about 27 mPa s, by measuring with a Bohlin Rheometer CVO, using geometry C25, and measuring the sample at 10° C. and at a shear rate of 50 s⁻¹.

TABLE 2
composition placebo

| Ingredient | Amount (g) |
|---|---|
| Demin water | 83.4 |
| HM pectin (AM 783, Danisco) | 0.4 |
| Sugar | 14.5 |
| Citric acid anhy. | 0.5 |
| Tripotassium citrate (IS 11655, Jungbunzlauer) | 0.54 |
| Carmoisine (311804SAM, Sensient) | 0.001 |
| Allura Red (312504SAM, Sensient) | 0.001 |
| Malt extract | 0.045 |
| Floral flavour | 0.21 |
| Total | 99.60 |

A parallel, double-blind, randomised, placebo-controlled healthy volunteer study is performed along the following. A population of 90 women aged 25-40 years is recruited and randomised in a parallel design into two balanced treatment groups; test (using the composition as in example 3 of table 1) and placebo (composition on table 2). Each treatment group is asked to consume one study drink (100 ml) per day for a duration of 12 weeks, with the main meal, e.g. the evening meal.

Subjects who are candidates for this evaluation will have skin types, commonly referred to Fitzpatrick skin types, of Category I-III according to the following definitions:
Category I Always burns easily, never tans
Category II Always burns easily, tans minimally
Category III Burns moderately, tans gradually (light brown)
Category IV Burns minimally, always tans well (moderate brown)
Category V Rarely burns, tans very well (moderate brown)
Category VI Never burns, deeply pigmented

Chroma-Meter Measurements

Changes in skin colour will be measured at two time points: at the start and at the end (after 12 weeks) of the trial. The Chromameter® will be used to perform this task. Measures will be taken on the palm of the hand, lower back and inner upper arm. Triplicate measurements will be taken on each site to obtain an average reading of the L*, a* and b* parameters. The measuring device will be placed gently on to the sites using minimal pressure to prevent blanching.

Colorimetric measurement of the skin will be done using a CR300 Minolta Chromameter® with an 8 mm head.

The Chromameter® converts colours perceived by man to a digital code composed of three parameters: L*: for clarity (from dark to light)
a*: for the green-to-red spectrum
b*: for the blue-to-yellow spectrum.

a* and b* are two chrominance parameters, L* is a luminance parameter.

The Chromameter® code enables highly accurate expression of skin colours and can thus distinguish between two skin areas, <<a priori>> appearing to be identical. After a calibration phase, measurements are done directly on the skin using a pulsed Xenon light source and a dual beam system designed to measure the light transmitted and to correct any slight deviation.

This instrument is commonly used for skin colour measurement in cosmetic and medical science and changes in b* value are known to be correlated with carotenoid accumulation in skin (Alaluf et al., 2002).

The results are set out in table 3. T0 is at the start of the trial, T12 is after 12 weeks usage.

TABLE 3

| | | | | | | | Change | | |
|---|---|---|---|---|---|---|---|---|---|
| | T0 | | | T12 | | | in b* (T12-T0) | | |
| b* | palm | back | arm | palm | back | arm | palm | back | arm |
| test | 11.12 ± 2.33 | 16.12 ± 2.40 | 13.2 ± 1.99 | 13.58 ± 1.89 | 15.91 ± 2.3 | 13.33 ± 1.9 | 2.46 | −0.21 | 0.13 |
| placebo | 10.51 ± 1.9 | 16.15 ± 2.59 | 14.05 ± 2.38 | 11.21 ± 1.99 | 14.7 ± 2.22 | 12.97 ± 1.89 | 0.7 | −1.45 | −1.08 |

For the change in b* value the p values were:

palm: p<0.0001

lower back: p<0.0001

arm: p<0.0003

The above results clearly show a change of skin colour (which is an indication of accumulation of the anti-oxidative carotenoids used in the formulation in the skin) but not so much as that the colouring would be too much.

1. Packaged liquid edible composition for treating the human skin by ingestion, said composition comprising a blend of at least two species of fruit and/or vegetables in liquified, puree, pulp, concentrate, extract or juice form, wherein said composition is packaged in a container in an amount of 50 to 150 g, and wherein said packaged composition comprises per container carotenoids in an amount of 10 to 14 mg, and which composition has a viscosity at 10° C. and at a shear rate of 50 s⁻¹ of 10 to 100 mPa s.

2. Composition according to claim 1, which comprises β-carotene in an amount of 5 to 8 mg, preferably 6 to 7.5 mg.

3. Composition according to claim 1, which comprises lycopene in an amount of 2 to 12 mg, preferably in an amount of 2.5 to 8 mg.

4. Composition according to claim 1, which comprises:
β-carotene in an amount of 5.5 to 8 mg, preferably 6.5 to 7.5 mg,
lycopene in an amount of 2 to 4 mg, preferably 2.5 to 3.5 mg,
lutein in an amount of 1 to 3 mg, preferably 1.5 to 2.5 mg.

5. Composition according to claim 1, wherein said composition has a Brix value of 14° to 21°, preferably 15.8° to 19.4°.

6. Composition according to claim 1, wherein said composition is packaged in a container in an amount of 75 to 125 g, more preferably 90-110 g.

7. Composition according to claim 1, which comprises a blend of at least one species of fruit in liquified, puree, pulp, concentrate, extract or juice form and at least one species of vegetables in liquified, puree, pulp, concentrate, extract or juice form.

8. Composition according to claim 1, which comprises orange carrot in liquified, puree, pulp, concentrate, extract or juice form, in an amount corresponding to fresh orange carrot of 40 to 200 g fresh weight, preferably 50 to 150 g fresh weight.

9. Composition according to claim 1, which comprises purple carrot in liquified, puree, pulp, concentrate, extract or juice form in an amount corresponding to fresh purple carrot of 20 to 150 g fresh weight, preferably 30 to 100 g fresh weight.

10. Composition according to claim 1, which comprises one or more of apple, white grape, red grape, banana, acerola cherry, lemon, orange, grapefruit, in liquified, puree, pulp, concentrate, extract or juice form.

11. Composition according to claim 1, wherein the composition comprises puree and/or juice and/or concentrate and/or juice concentrate of fruit and/or vegetable origin in an amount corresponding to 200-400 g, preferably 250-350 g fresh weight of fruit and/or vegetables.

12. Composition according to claim 1, wherein the composition has a viscosity of 15 to 90, more preferably 20 to 60 mPa s.

13. Method for treating the human skin by ingestion of a composition according to claim 1.

14. Method according to claim 13, wherein treating the human skin comprises one or more of skin care, skin condition, skin appearance, skin colour, for a skin bronzing, skin browning, skin colouring effect, UV-protection, UV-B-protection, sun-screen, protection against aging, providing anti-oxidants to the skin, protection against photo-induced aging.
